(19) United States
(12) Patent Application Publication
(10) Pub. No.: US 2025/0011877 A1
(43) Pub. Date: Jan. 9, 2025

(54) MITOCHONDRIAL GENOTYPICAL SCORES: PROGNOSTIC MARKERS IN CHEMOSENSITIVE CANCER

(71) Applicants: UNIVERSITE GRENOBLE ALPES, Saint Martin d'Heres (FR); CENTRE HOSPITALIER REGIONAL DE GRENOBLE, La Tronche (FR); CENTRE NATIONAL DE LA RECHERCHE SCIENTIFIQUE, Paris (FR); INSTITUT NATIONAL DE LA SANTE ET DE LA RECHERCHE MEDICALE, Paris (FR)

(72) Inventors: Julie MONDET, La Tronche (FR); Pascal MOSSUZ, La Tronche (FR); Baptiste LAC, La Tronche (FR)

(73) Assignees: UNIVERSITE GRENOBLE ALPES, Saint Martin d'Heres (FR); CENTRE HOSPITALIER REGIONAL DE GRENOBLE, La Tronche (FR); CENTRE NATIONAL DE LA RECHERCHE SCIENTIFIQUE, Paris (FR); INSTITUT NATIONAL DE LA SANTE ET DE LA RECHERCHE MEDICALE, Paris (FR)

(21) Appl. No.: 18/710,819

(22) PCT Filed: Nov. 16, 2022
(86) PCT No.: PCT/FR2022/052098

§ 371 Date: May 16, 2024

(30) Foreign Application Priority Data
Nov. 16, 2021 (FR) FR2112107

(51) Int. Cl. C12Q 1/6886

(52) U.S. Cl. CPC C12Q 1/6886; C12Q 2600/106; C12Q 2600/118; C12Q 2600/156

(57) ABSTRACT

A prognostic risk score for chemosensitive cancer, in particular acute myeloid leukemia (AML), based on somatic genetic abnormalities affecting certain genes of the mitochondrial genome.

### TECHNICAL FIELD OF THE INVENTION

The present invention concerns a prognostic risk score for chemosensitive cancer, in particular acute myeloid leukemia (AML), based on somatic genetic abnormalities affecting certain genes of the mitochondrial genome.

In the description below, references in parentheses [ ] refer to the list of references presented at the end of the text.

### STATE OF THE ART

The acute myeloid leukemias (AML) are uncontrolled malignant proliferations of immature myeloid progenitors called leukemia cells stuck in an early stage of differentiation, being accumulated in the bone marrow and eventually in other organs. Despite therapeutic progress, their prognosis is very poor with a recovery in 35 to 40% of adult patients aged under 60 years and in 5 to 15% of patients aged over 60 years. The prognostic factors currently used in practice are based on age, the level of circulating white blood cells, the European LeukemiaNet prognostic classification (ELN) 2017 (Dohner et al., 2017) [1]. The ELN 2017 classification is based on cytogenetic and/or molecular alterations and defines three statuses respectively: favorable, intermediate and unfavorable. Nonetheless, this prognostic classification remains to be improved. For example, in the favorable group, the 5-year survival rate is only 64% in patients under 60 years old (35% in those over 60 years old) [2]. There is a recent update to the European LeukemiaNet prognostic classification: the ELN 2022 (Dohner et al., 2022) [3].

The mitochondria are cellular organelles of bacterial origin present in all eukaryotic cells (except red blood cells). They play an essential role in the regulation of cellular metabolism, in energy supply (Krebs cycle, β-oxidation of fatty acids, etc.), but also in calcium homeostasis, the generation of reactive oxygen species (ROS) and in the triggering of apoptosis phenomena. The somatic mutations of isocitrate dehydrogenases (IDH1, IDH2), mitochondrial enzymes present on the nuclear genome, lead to deregulation of the Krebs cycle with the accumulation of a neometabolite, 2-hydroxyglutarate, which contributes to leukemogenesis.

In AML, the level of activation of oxidative phosphorylation (OXPHOS) is directly correlated with the resistance of leukemia cells (Farge et al, 2017) [4]. In addition, transfers of mitochondria between stromal cells and leukemic cells have been described as responsible for the resistance of leukemic cells to chemotherapy. In a prospective study (DRCI LAM38RC13-209), it was shown that a deregulation of the production of reactive oxygen species (ROS) by the mitochondria of leukemia cells was associated with a poor prognosis (decrease in overall survival) independently of the usual prognostic factors for AML (age, WBC/I, ELN 2017, transplant) (Mondet et al, 2019) [5]. Thus, several avenues converge on the role of the mitochondria in the chemoresistance of AML.

Several publications even suggest the use of drugs targeting the mitochondria in AML or in other cancers in order to limit the acquisition of resistance to chemotherapies and increase sensitivity to them (Bosc et al., 2021; Neuzil et al., 2013) [6, 7]. However, currently there are no specific companion tests for these new therapeutic strategies which only provide benefit in certain patients. A companion test is a diagnostic test that makes it possible to select, based on their status for a predictive marker identified by this test, only those patients in whom the treatment is likely to provide a benefit among those diagnosed for a given disease.

The venetoclax is an inhibitor of the anti-apoptotic protein BCL-2, a protein localized at the mitochondrial membrane. The venetoclax is bonded directly to the binding groove of the BH3 domain of BCL-2, by displacing the pro-apoptotic proteins containing the BH3 motif such as BIM, to initiate mitochondrial outer membrane permeabilization (MOMP), the caspase activation and the apoptosis. This treatment is currently authorized for use in chronic lymphocytic leukemia, the lymphomas and recently in the indication Acute myeloid leukemia in «unfit» patients, that is to say those unable to tolerate standard chemotherapy. The response to treatment with the venetoclax is linked to the state of the mitochondria. But no link with mutations in the mitochondrial genome has currently been described.

Indeed, regarding the mitochondrial genome, few studies have analyzed the impact of mutations in the mitochondrial genome as a prognostic marker in AML. The ND4 mutations, encoding a subunit of complex I, were analyzed in 452 patients via a targeted sequencing approach (different from high-throughput sequencing technology) in AML and are associated with increased overall survival in a univariate analysis (Damm et al., 2012) [8]. However, the multivariate analysis does not significantly confirm this result (p=0.089). The article even concludes that this result should be confirmed with other studies. Another study investigated the prognostic impact of ND4 mutations in 121 AML patients (Chun et al., 2014) [9]. However, the study shows no difference in terms of overall survival or relapse-free survival. The prognostic impact of ND4 is therefore not a certain data for those skilled in the art.

The mutations affecting the COX1 and COX2 genes were analyzed by high-throughput sequencing (Silkjaer et al., 2013) [13] and by Sanger sequencing in acute myeloid leukemia (Silkjaer et al., 2013) [14]. In the high-throughput sequencing study, COX1 and COX2 mutations were associated with decreased overall survival in univariate analysis. However, the multivariate analysis does not significantly confirm this result [13]. In the subsequent Sanger sequencing study, in the overall group of 165 affected patients treated with curative chemotherapy, there was no prognostic impact of the presence of COX1 or COX2 variations [14]. The prognostic impact of COX1 and COX2 mutations is therefore not a certain data for those skilled in the art. In addition, the technique used by Sanger type sequencing has a sensitivity threshold of 20% (see material and methods of the publication, [14]) which underestimates the detection of variants in the cohort of patients (16% of variants in the COX1 and COX2 genes versus approximately 30-35% by a high-throughput sequencing technique taking into account a heteroplasmy threshold 2%). Finally, the high-throughput sequencing study did not take haplogroups into account in the selection of variants.

Nonetheless, the mitochondrial genome comprises 16 kB whose 37 genes coding for 13 proteins involved in the respiratory chain as well as 22 tRNAs and 2 rRNAs. Another study based on public somatic genetic data taken from the cbioportal site (https://www.cbioportal.org/) analyzed the impact of mutations on acute myeloid leukemia (Wu et al., 2018) [10]. Nonetheless, several biases exist in this study. One of them is linked to the use of bioinformatics analysis dedicated to genomic DNA, not specific to mitochondrial DNA. In this study, only 8% of AML patients have mutations present in genes (ND1, ND2, ND3, ND4, ND4L, ND5, ND6, CYB, COX1, COX2, COX3, ATP6, ATP8). The error in analyzing genetic data leads to an underestimate of the number of variants distorting the final results. For comparison, in the study targeting only the ND4 gene (Damm et al., 2012) [8], the frequency of mutations was 6.4% (29/452). Since mitochondrial DNA has its own genetic code, using a common bioinformatics analysis between nuclear genomic mutations and mitochondrial mutations causes errors in the interpretation of the obtained variants (Caudron-Herger and Diederichs, 2018) [11]. The same analysis bias exists in other pathologies (e.g. cbioportal site dated Aug. 10, 2021, Diffuse Large B cell Lymphoma study, Duke 2017, 0% presents mutations in the ND1, ND2, ND3, ND4, ND4L genes vs 35% in the study by Zeng et al., 2018) [12]).

Apart from the studies by Damm et al. and Chun et al. analyzing only the ND4 gene, by Silkjaer et al. analyzing the COX1 and COX2 genes [13-14], and WU et al. using inappropriate bioinformatics analysis, no study has shown the contribution of mitogenome mutations in the stratification of AML patients.

Consequently, the study of the mitochondrial genome and the identification of possible new prognostic markers for AML remains a challenge, in order to better classify patients suffering from AML in terms of overall survival, and thus adapt their therapeutic management according to their survival prognosis.

### DESCRIPTION OF THE INVENTION

The inventors are the very first to have demonstrated a score based on combinations of presence and absence of mutations in certain genes of the mitochondrial genome as prognostic markers for acute myeloid leukemia (AML).

The inventors have thus developed a score, hereinafter called Mitoscore, to predict the response to chemotherapy treatment and the survival of patients suffering from AML. The Mitoscore is based on mitochondrial genome sequencing technology (for example by NGS technology or other). The Mitoscore based in particular on molecular abnormalities of the genes ND1, ND2, ND3, ND4, ND5, CYTB, ATP6, ATP8, MOX, COX2, OX3, 12S (otherwise named MT-ND1, MT-ND2, MT-ND3, MT-ND4, MT-ND5, MT-CYB, MT-ATP6, MT-ATP8, MT-CO1, MT-CO2, MT-CO3, MT-RNR1, respectively; see table 1 below indicating the official nomenclature of the mitochondrial genes used in the prognostic scores of the invention) stratifies at diagnosis patients suffering from AML into 3 respective prognosis groups: favorable, intermediate, unfavorable, in terms of overall survival on the basis of combinations of variants of the genes above.

TABLE 1

| | HGNC | NCBI Gene ID | Alias |
|---|---|---|---|
| MT-CO1 | 7419 | 4512 | COX1 |
| MT-CO2 | 7421 | 4513 | COX2 |
| MT-CO3 | 7422 | 4514 | COX3 |
| MT-RNR1 | 7470 | 4549 | 12S or MOTS-c |
| MT-ND4 | 7459 | 4538 | ND4 |
| MT-ND2 | 7456 | 4536 | ND2 |
| MT-ND3 | 7458 | 4537 | ND3 |
| MT-ND5 | 7461 | 4540 | ND5 |
| MT-ATP8 | 7415 | 4509 | ATP8 |
| MT-ATP6 | 7414 | 4508 | ATP6 |
| MT-ND1 | 7455 | 4535 | ND1 |
| MT-CYB | 7427 | 4519 | CYTB |

The Mitoscore allows an improved prognostic stratification compared to the European LeukemiaNet prognostic score 2017 (ELN 2017) currently used for patients suffering from AML, in order to distinguish good responders from poor responders and to adapt the therapeutic management. The Mitoscore also allows for improved prognostic stratification compared to the recently published prognostic score European LeukemiaNet 2022 (ELN 2022). The Mitoscore is valid independently of the usual prognostic factors (age, white blood cell count, ELN, transplant), and can be combined with the prognostic EuroLeukemiaNet 2017 (ELN 2017) or EuroLeukemiaNet 2022 (ELN 2022)—generically called ELN prognostic score—in the form of a score, hereinafter referred to as Mitoscore+.

The Mitoscore improves the prognostic stratification of patients with AML at the diagnosis. In addition, the Mitoscore carried out by the sequencing the mitochondrial genome (16 kb) proves to be simpler than the classification of the ELN requiring both the culturing of leukemic cells for the production of a karyotype and a sequencing of the molecular anomalies for example by NGS technique (panel of approximately 100 kb, different depending on the center). In addition, the karyotype requires a significant number of leukemic cells not always obtained in poor marrows.

The Mitoscore therefore saves time, reduces costs, easier reproducibility (no culturing, no interpretation difficulties), and requires a lower quantity of biological material.

The subject of the present invention is therefore an in vitro method for establishing a Mitoscore survival prognosis in a patient suffering from chemosensitive cancer, said method comprising the following steps:
detecting in a biological sample of said patient, the presence and/or absence of at least one mutation on one of the following nine genes of the mitochondrial genome: ND1, ND2, ND3, ND4, ND5, CYTB, ATP8, ATP6, COX1, COX2, COX3 and 12S;
comparing the detection results compared to a reference biological sample or a reference sequence or a reference haplogroup/haplotype;
establishing the survival prognosis of said patient.

The term «chemosensitive cancer» within the meaning of the present invention means a pathology that can be treated by chemotherapy (e.g. anthracyclines, antimetabolics, alkaloids or topoisomerase inhibitors, alone or in combination with other treatments) selected for example from the group consisting of acute myeloid leukemia (AML), sarcomas, testicular cancer (germ cell cancer in general), choriocarcinoma, hemopathy, gynecological cancer such as ovarian cancer, and breast cancer, lung cancer, neuroblastomas, malignant brain tumors, digestive cancers, pancreatic cancers, bladder cancers, prostate cancers, thyroid cancers, liver cancers, ENT cancers. Preferably, it is acute myeloid leukemia (AML).

The term «biological reference sample» within the meaning of the present invention means a biological sample from a healthy subject, for example a DNA sample from marrow, blood, or tissue.

The term «reference sequence» within the meaning of the present invention means a reference mitochondrial DNA sequence, for example the rCRS sequence (revised Cambridge Reference Sequence NC_012920.1) or a sequence present in the Mitomap/Mitomaster databases, gnomAD.

The term «reference haplogroup/haplotype» according to the present invention means the variations in composition relative to the reference sequence (CRS or rCRS) defining a haplogroup or haplotype according to the classification of Richards and Macaulay of 1998, which can be determined by several tools (MitoTool, HaploFind, PhyloTree mt, Mitomap, . . . ).

According to a particular embodiment of the in vitro method for establishing a Mitoscore survival prognosis in a patient suffering from chemosensitive cancer according to the present invention:
the presence of at least one mutation in one of the ND2, ND3, ATP8, CYTB, ND4 genes and the absence of mutation in the COX1, COX2, COX3, 12S genes, is an indication of a prognosis of favorable survival;
the presence of at least one mutation in one of the COX1, COX2, COX3, 12S genes and the absence of mutations in the ND2, ND3, ATP8, CYTB, ND4 genes is an indication of a prognosis of unfavorable survival; and
either the absence of mutations in the ND2, ND3, ATP8, CYTB, ND4, COX1, COX2, COX3, 12S genes (patients called «Mitonaïf A», corresponding to approximately 30% of patients with AML), or the presence of at least one mutation in one of the genes ND2, ND3, ATP8, CYTB, ND4 and at least one mutation in one of the genes COX1, COX2, COX3, 12S, is an indication of an intermediate survival prognosis.

According to a particular embodiment of the in vitro method for establishing a Mitoscore survival prognosis in a patient suffering from chemosensitive cancer according to the present invention:
the presence of at least one mutation in one of the ND2, ND3, ATP8, CYTB genes, and the absence of mutations in the COX1, COX2, COX3, 12S genes, is an indication of a favorable survival prognosis;
the presence of at least one mutation in one of the COX1, COX2, COX3, 12S genes and the absence of mutations in the ND2, ND3, ATP8, CYTB, ND4 genes is an indication of an unfavorable survival prognosis; and
either the absence of mutations in the ND2, ND3, ATP8, CYTB, ND4, COX1, COX2, COX3, 12S genes (patients called «Mitonaïf A», corresponding to approximately 30% of patients with AML), or the presence of at least one mutation in one of the ND2, ND3, ATP8, CYTB, ND4 genes and at least one mutation in one of the COX1, COX2, COX, 12S genes, is an indication of an intermediate survival prognosis.

According to a particular embodiment of the in vitro method for establishing a Mitoscore B survival prognosis in a patient suffering from chemosensitive cancer according to the present invention:
the presence of at least one mutation in one of the ND2/ND5/ATP6/CYTB/ND4 genes, and the absence of mutation in the following genes ND1/COX3/12S, is the indication of a favorable survival prognosis;
the presence of at least one mutation in one of the ND1/COX3/12S genes, and the absence of mutation in the following genes ND2/ND5/ATP6/CYTB/ND4, is an indication of an unfavorable survival prognosis; and
either the absence of mutations in the ND2/ND5/ATP6/CYTB/ND4/ND1/COX3/12S genes (patients called «Mitonaïf B», corresponding to approximately 40% of patients with AML), or the presence of at least one mutation in one of the ND2/ND5/ATP6/CYTB/ND4 genes and at least one mutation in one of the ND1/COX3/12S genes (patients called ((mutated intermediate»), is the indication of an intermediate survival prognosis.

The term «favorable», «intermediate» or «unfavorable» survival prognosis means within the meaning of the present invention, the graduation of the hypotheses made on the evolution of the pathology on the basis of mitochondrial variants (i.e. in terms of chances of survival, risk of complications and/or death).

The present invention also relates to an in vitro method for establishing a Mitoscore+ survival prognosis in a patient suffering from chemosensitive cancer not having any mutation in the «ND2, ND3, ATP8, CYTB, ND4, COX1, COX2, COX3, 12S» or «ND2, ND5, ATP6, CYTB, ND4, ND1, COX3, 12S» genes (i.e. patients of sub-groups called Mitonaïf A and Mitonaïf B, respectively), said method comprising the method for establishing a Mitoscore survival prognosis as defined above and determining the ELN prognostic score, in said patient. In the subgroups of patients called Mitonaïf A or Mitonaïf B of the «intermediate» Mitoscore, the ELN stratification was applied to determine the Mitoscore+ which made it possible to reclassify these patients with AML into 3 prognosis groups in terms of overall survival.

According to a particular embodiment of an in vitro method for establishing a Mitoscore+ survival prognosis in a patient suffering from chemosensitive cancer according to the present invention:
either the presence of at least one mutation in one of the ND2/ND3/ATP8/CYTB/ND4 genes and the absence of mutation in the COX1/COX2/COX3/12S genes, or the absence of mutations in the ND2, ND3, ATP8, CYTB, ND4, COX1, COX2, COX3, 12S genes and a «favorable» classification according to the ELN score, is an indication of a favorable survival prognosis;
either the presence of at least one mutation in one of the COX1/COX2/COX3/12S genes and the absence of mutation in the ND2/ND3/ATP8/CYTB/ND4 genes, or the absence of mutations in the ND2, ND3, ATP8, CYTB, ND4, COX1, COX2, COX3, 12S genes and an «unfavorable» classification according to the ELN score, is an indication of an unfavorable survival prognosis;
either the presence of at least one mutation in one of the ND2/ND3/ATP8/CYTB/ND4 genes and at least one mutation in one of the COX1/COX2/COX3/12S genes, or the absence mutations in the ND2, ND3, ATP8, CYTB, ND4, COX1, COX2, COX3, 12S genes and an «intermediate» classification according to the ELN score, is an indication of an intermediate survival prognosis.

According to a particular embodiment of an in vitro method for establishing a Mitoscore+ survival prognosis in a patient suffering from chemosensitive cancer according to the present invention:
either the presence of at least one mutation in one of the ND2/ND3/ATP8/CYTB genes and the absence of mutation in the COX1/COX2/COX3/12S genes, or the absence of mutations in the ND2, ND3, ATP8, CYTB, ND4, COX1, COX2, COX3, 12S genes and a «favorable» classification according to the ELN score, is an indication of a favorable survival prognosis;
either the presence of at least one mutation in one of the COX1/COX2/COX3/12S genes and the absence of mutation in the ND2/ND3/ATP8/CYTB/ND4 genes, or the absence of mutations in the ND2, ND3, ATP8, CYTB, ND4, COX1, COX2, COX3, 12S genes and an «unfavorable» classification according to the ELN score, is an indication of an unfavorable survival prognosis;
either the presence of at least one mutation in one of the ND2/ND3/ATP8/CYTB/ND4 genes and at least one mutation in one of the COX1/COX2/COX3/12S genes, or the absence mutations in the ND2, ND3, ATP8, CYTB, ND4, COX1, COX2, COX3, 12S genes and an «intermediate» classification according to the ELN score, is an indication of an intermediate survival prognosis.

According to a particular embodiment of an in vitro method for establishing a Mitoscore B+ survival prognosis in a patient suffering from chemosensitive cancer according to the present invention:
either the presence of at least one mutation in one of the ND2/ND5/ATP6/CYTB/ND4 genes and the absence of mutation in the following genes ND1/COX3/12S, or the absence of mutations in the ND2, ND5, ATP6, CYTB, ND4, ND1, COX3, 12S genes and a «favorable» classification according to the ELN score, is an indication of a favorable survival prognosis;
either the presence of at least one mutation in one of the ND1/COX3/12S genes and the absence of mutation in the following genes ND2/ND5/ATP6/CYTB/ND4, or the absence of mutations in the ND2, ND5, ATP6, CYTB, ND4, ND1, COX3, 12S genes and an «unfavorable» classification according to the ELN score, is an indication of an unfavorable survival prognosis;
either the presence of at least one mutation in one of the ND2/ND5/ATP6/CYTB/ND4 genes and at least one mutation in one of the ND1/COX3/12S genes, or the absence of mutations in the ND2, ND5, ATP6, CYTB, ND4, ND1, COX3, 12S genes and an «intermediate» classification according to the ELN score, is an indication of an intermediate survival prognosis.

The present invention also relates to an in vitro method for establishing a Mitoscore B/A survival prognosis in a patient suffering from a chemosensitive cancer, in particular acute myeloid leukemia (AML), according to the present invention, where:
the indication of a favorable survival prognosis corresponds either to the presence of at least one mutation in one of the ND2/ND5/ATP6/CYTB/ND4 genes, and the absence of mutations in the following genes ND1/COX3/12S, namely «the absence of mutations in the ND2/ND5/ATP6/CYTB/ND4/ND1/COX3/12S genes and the presence of at least one mutation in one of the «ND3, ATP8» genes and the absence of mutations in the «COX1, COX2» genes;
the indication of an unfavorable survival prognosis corresponds either to the presence of at least one mutation in one of the ND1/COX3/12S genes, and the absence of mutation in the following genes ND2/ND5/ATP6/CYTB/ND4, or the absence of mutations in the ND2/ND5/ATP6/CYTB/ND4/ND1/COX3/12S genes and the presence of at least one mutation in one of the «COX1, COX2» and the absence of mutations in one of the «ND3, ATP8» genes;
the indication of an intermediate survival prognosis corresponds either to the presence of at least one mutation in one of the ND2/ND5/ATP6/CYTB/ND4 genes and at least one mutation in one of the genes ND1/COX3/12S or the absence of mutations in the ND2/ND5/ATP6/CYTB/ND4/ND1/COX3/12S genes and the presence of at least one mutation in one of the «COX1, COX2» genes and the presence of at least one mutation in one of the «ND3, ATP8» genes or the absence of mutations in the ND2/ND5/ATP6/CYTB/ND4/ND1/COX3/12S/COX1/COX2/ND3/ATP8 genes (patients called Mitonaives B/A, namely approximately 25% of AML patients).

The present invention also relates to an in vitro method for establishing a Mitoscore B/A+ survival prognosis in a patient suffering from chemosensitive cancer, in particular acute myeloid leukemia (AML), according to the present invention, where:
the indication of a favorable survival prognosis corresponds to either the presence of at least one mutation in one of the ND2/ND5/ATP6/CYTB/ND4 genes, and the absence of mutation in the following genes ND1/COX3/12S, or «the absence of mutations in the ND2/ND5/ATP6/CYTB/ND4/ND1/COX3/12S genes and the presence of at least one mutation in one of the «ND3, ATP8» genes and the absence of mutations in the «COX1, COX2» genes, or in Mitonaive B/A patients classified as favorable with the ELN classification;
the indication of an unfavorable survival prognosis corresponds either to the presence of at least one mutation in one of the ND1/COX3/12S genes, and the absence of mutation in the following genes ND2/ND5/ATP6/CYTB/ND4, or the absence of mutations in the ND2/ND5/ATP6/CYTB/ND4/ND1/COX3/12S genes and the presence of at least one mutation in one of the «COX1, COX2» genes and the absence of mutations in the «ND3, ATP8» genes or in Mitonaive B/A patients classified unfavorable with the ELN classification;
the indication of an intermediate survival prognosis corresponds either to the presence of at least one mutation in one of the ND2/ND5/ATP6/CYTB/ND4 genes and at least one mutation in one of the ND1/COX3/12S genes or the absence of mutations in the ND2/ND5/ATP6/CYTB/ND4/ND1/COX3/12S genes and the presence of at least one mutation in one of the «COX1, COX2» genes and the presence of at least one mutation in one of the «ND3, ATP8» genes, or for Mitonaive B/A patients classified intermediate with the ELN classification.

The present invention also relates to an in vitro method for establishing a Mitoscore A/B survival prognosis in a patient suffering from chemosensitive cancer, in particular acute myeloid leukemia, according to the present invention, where:
the indication of a favorable survival prognosis corresponds either to the presence of at least one mutation in one of the ND2, ND3, ATP8, CYTB, ND4 genes and the absence of mutation in the COX1, COX2, COX3, 12S genes, or the absence of mutations in the COX1, COX2, COX3, 12S, ND2, ND3, ATP8, CYTB, ND4 genes but the presence of at least one mutation in one of the ATP6, ND5 genes and the absence of mutations in the ND1 gene;
the indication of an unfavorable survival prognosis corresponds either to the presence of at least one mutation in one of the COX1, COX2, COX3, 12S genes and the absence of mutations in the ND2, ND3, ATP8, CYTB, ND4 genes, or the absence of mutations in the COX1, COX2, COX3, 12S, ND2, ND3, ATP8, CYTB, ND4 genes but the presence of at least one mutation in the ND1 gene and the absence of mutations in one of the ATP6, ND5 genes;
the indication of an intermediate survival prognosis corresponds to either the presence of at least one mutation in one of the ND2, ND3, ATP8, CYTB, ND4 genes and at least one mutation in one of the COX1, COX2, COX3, 12S genes, or the absence of mutations in the ND2, ND3, ATP8, CYTB, ND4, COX1, COX2, COX3, 12S genes, and the presence of at least one mutation in one of the ND5/ATP6 genes and the presence of at least one mutation in the ND1 gene or the absence of mutations in the ND2, ND3, ATP8, CYTB, ND4, COX1, COX2, COX3, 12S, ND5, ATP6, ND1 genes (patients called Mitonaive A/B namely approximately 25% of patients in AML).

The present invention also relates to an in vitro method for establishing a Mitoscore A/B+ survival prognosis in a patient suffering from chemosensitive cancer, in particular an acute myeloid leukemia, according to the present invention, where:
the indication of a favorable survival prognosis corresponds either to the presence of at least one mutation in one of the ND2, ND3, ATP8, CYTB, ND4 genes and the absence of mutations in the COX1, COX2, COX3, 12S genes, or the absence of mutations in the COX1, COX2, COX3, 12S, ND2, ND3, ATP8, CYTB, ND4 genes but the presence of at least one mutation in one of the ATP6, ND5 genes and the absence of mutations in the ND1 gene or in Mitonaive A/B patients classified favorable with the ELN classification;
the indication of an unfavorable survival prognosis corresponds either to the presence of at least one mutation in one of the COX1, COX2, COX3, 12S genes and the absence of mutations in the ND2, ND3, ATP8, CYTB, ND4 genes, or the absence of mutations in the COX1, COX2, COX3, 12S, ND2, ND3, ATP8, CYTB, ND4 genes but the presence of at least one mutation in the ND1 gene and the absence of mutations in one of the ATP6, ND5 genes or in Mitonaive A/B patients classified unfavorable with the ELN classification;
the indication of an intermediate survival prognosis corresponds to either the presence of at least one mutation in one of the ND2, ND3, ATP8, CYTB, ND4 genes and at least one mutation in one of the COX1, COX2, COX3, 12S genes, or the absence of mutations in the ND2, ND3, ATP8, CYTB, ND4, COX1, COX2, COX3, 12S genes, and the presence of at least one mutation in one of the ND5/ATP6 genes and the presence of at least one mutation in the ND1 gene or Mitonaive A/B patients classified intermediate with the ELN classification.

By «favorable», «intermediate» or «unfavorable» survival prognosis is meant within the meaning of the present invention, the graduation of the hypotheses made on the evolution of the pathology on the basis of mitochondrial variants (i.e. in terms of chances of survival, risk of complications and/or death).

According to a particular embodiment of a method according to the present invention, the detection steps are carried out by high-throughput sequencing (NGS) of the mitochondrial genome.

The present invention also relates to an in vitro method for predicting or evaluating the effectiveness and/or benefit of a treatment of a chemosensitive cancer, in particular of acute myeloid leukemia (AML), in a patient suffering from said cancer comprising the following steps:
determination of a survival prognosis Mitoscore, Mitoscore+, Mitoscore B, Mitoscore B+, Mitoscore A/B, Mitoscore A/B+, Mitoscore B/A or Mitoscore B/A+ according to a method according to the present invention from a biological sample from said patient before treatment;
determination of a survival prognosis Mitoscore, Mitoscore+, Mitoscore B, Mitoscore B+, Mitoscore A/B, Mitoscore A/B+, Mitoscore B/A or Mitoscore B/A+ according to a method according to the present invention from a biological sample from said patient after treatment;
comparison of survival prognoses Mitoscore, Mitoscore+, Mitoscore B, Mitoscore B+, Mitoscore A/B, Mitoscore A/B+, Mitoscore B/A or Mitoscore B/A+ before and after treatment;
determination of either a resistance to treatment when the survival prognosis of said patient after treatment is identical to or worse than the survival prognosis of said patient before treatment, or a sensitivity to treatment when the survival prognosis of said patient after treatment is better than the survival prognosis of said patient before treatment.

The term «chemosensitive cancer» within the meaning of the present invention means a pathology that can be treated by chemotherapy (e.g. anthracyclines, antimetabolics, alkaloids or topoisomerase inhibitors, alone or in combination with other treatments) selected for example from the group consisting of the acute myeloid leukemias (AML), sarcomas, testicular cancer (germ cell cancer in general), choriocarcinoma, hemopathy, gynecological cancer such as ovarian cancer, and breast cancer, lung cancer, neuroblastomas, malignant brain tumors, digestive cancers, pancreatic cancers, bladder cancers, prostate cancers, thyroid cancers, liver cancers, ENT cancers.

The term «identical survival prognosis before and after treatment» within the meaning of the present invention means a favorable, intermediate or unfavorable survival prognosis before treatment which remains, respectively, a favorable, intermediate or unfavorable survival prognosis after treatment.

The term «poorer survival prognosis after treatment than before treatment» within the meaning of the present invention means, for example, a favorable or intermediate survival prognosis before treatment which becomes, respectively, an intermediate or unfavorable prognosis after treatment.

The term «better survival prognosis after treatment than before treatment» within the meaning of the present invention means, for example, an intermediate or unfavorable survival prognosis before treatment which becomes, respectively, a favorable or intermediate survival prognosis after treatment.

According to a particular embodiment of the method of the present invention, the treatment of a chemosensitive cancer, in particular of acute myeloid leukemia, comprises the administration of venetoclax possibly in combination with another molecule (e.g. azacytidine).

A computer program was created for implementing the above methods. This analyzes the variants from the source data of the used sequencer, the filters according to quality criteria and the location of the variants in the coding zones, the heteroplasmy rate and silent mutations. The program subsequently applies the Mitoscores of the invention (Mitoscore/Mitoscore+/Mitoscore B/Mitoscore B+, Mitoscore B/A, Mitoscore B/A+, Mitoscore A/B, Mitoscore A/B+).

The present invention therefore also relates to a computer program including instructions, which when executed by a computer, lead it to implement the methods described above.

Thus, the implementation of Mitoscores is carried out thanks to said computer program developed to analyze the sequences of the mitochondrial genome.

The present invention also relates to a data carrier readable by a computer including instructions which, when executed by a computer, lead it to implement a method as described above. In one embodiment, said data carrier is non-transient.

The present invention also relates to a method making it possible to select, depending on their status for a predictive marker identified by this test, only those patients in whom the treatment is likely to bring a benefit among those diagnosed for a given disease and thus to predict the addition of molecules/drugs targeting the mitochondria in chemosensitive cancers, in particular in acute myeloid leukemia. For example: patients in the unfavorable or intermediate group of Mitoscores presenting mutations in the 12S gene can be treated with the MOTS-c peptide in order to improve the response to the reference treatment; patients in the unfavorable or intermediate group of Mitoscores with mutations in the COX1, COX2 or COX3 genes can be treated with quercetin or a drug acting on the complex IV in order to improve the response to treatment; patients in the intermediate or unfavorable Mitoscore group may benefit from therapies targeting complexes I or III of the respiratory chain (e.g. Olaparib, Mubritinib, trimetazidine dihydrochloride, etc.) in addition to the standard treatment.

The present invention therefore relates to the use of a method according to the present invention to establish a Mitoscore in a patient suffering from a chemosensitive cancer as a companion test, in particular a companion test to the addition of molecules targeting the mitochondria.

### BRIEF DESCRIPTION OF THE FIGURES

FIG. 1 represents the technical workflow carried out for the application of Mitoscore and Mitoscore+, defining in particular the criteria used for the selection of variants.

FIG. 2 represents the Kaplan-Meier survival curves (log-rank test p<0.05) representing the overall survival as a function of time in patients with AML according to (A) the Mitoscore or (B) the Mitoscore+.

FIG. 3 represents the Hazard Ratio (HR) (95% CI) of the risk of death according to the Mitoscore and the Mitoscore+ calculated according to the Cox model in univariate (UV) or multivariate (MV) analysis. The covariates used in the MV analysis are age at diagnosis, white blood cell (WBC) count in G/l, the ELN 2017 classification and/or bone marrow transplant.

FIG. 4 represents the Kaplan-Meier survival curves (log-rank test p<0.05) representing the overall survival as a function of time in AML patients according to (A) the Mitoscore B containing the ND2/ND5/CYTB/ND4/ATP6/ND1/CO3/12S genes or (B) the Mitoscore B+ containing the ND2/ND5/CYTB/ND4/ATP6/ND1/CO3/12S genes associated with ELN 2017 in Mitonaive patients for the genes previously cited.

FIG. 5 represents the Hazard Ratio (HR) (95% CI) of the risk of death according to the Mitoscore B and the Mitoscore B+ calculated according to the Cox model in univariate (UV) or multivariate (MV) analysis. The covariates used in the MV analysis are age at diagnosis, white blood cell (WBC) count in G/l, the ELN 2017 classification and/or bone marrow transplant.

FIG. 6 is a block diagram representing a computer for implementing a prognosis establishment method according to the invention.

FIG. 7 represents the Kaplan-Meier survival curves (log-rank test p<0.05) representing the overall survival as a function of time in patients with AML according to (A) the Mitoscore B/A or (B) the Mitoscore B/A+ associated with ELN 2017 in Mitonaive B/A patients.

FIG. 8 represents the Kaplan-Meier survival curves (log—rank test p<0.05) representing the overall survival as a function of time in patients with AML according to (A) the Mitoscore A/B or (B) the Mitoscore A/B+ associated with the ELN 2017 in Mitonaive A/B patients.

### EXAMPLES

### Example 1: Development of Mitochondrial Genotypical Scores in the Context of Prognosis of Acute Myeloid Leukemia (AML)

Summary: the inventors sequenced the entire mitochondrial genome using high-throughput mitochondrial genome sequencing (NGS) technology from patients suffering from AML. From this sequencing, a score called Mitoscore was defined which made it possible to predict patient survival. The benefit of Mitoscore was confirmed in multivariate analysis independently of the usual prognostic factors for AML (i.e. age, number of circulating white blood cells (WBC), cytogenetic and molecular abnormalities, transplant). Moreover, the Mitoscore was functionally characterized by comparison with ROS emission data with and without inhibitors of mitochondrial complexes (antimycin A, Rotenone).

### Definition of the Retained Variants:

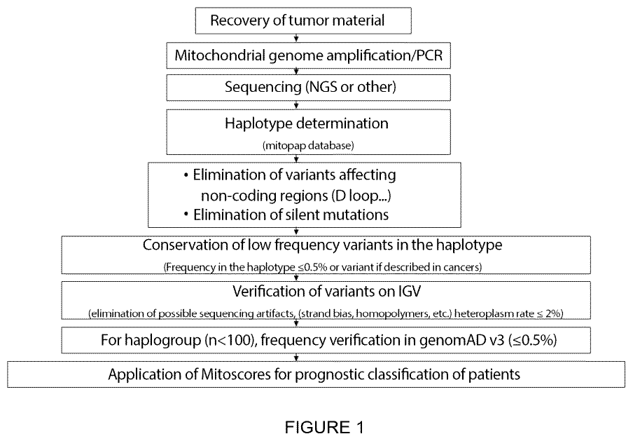

The sequencing of the mitochondrial genome was carried out on a machine S5 (Ion Torrent) after PCR amplification of the mitochondrial genome (2 fragments of 8 kDa). The mitochondrial genome can be made with other technologies for library synthesis, for example PCR amplification of the mitochondrial genome with more than 2 fragments or recovery of the sequencing product by capture. In addition, the sequencing on an Illumina technology type sequencer can also be carried out. Variant analysis was performed via Mitomaster. Variants affecting non-coding regions (e.g. D—loop, etc.), heteroplasmy rates strictly below 2% and silent mutations were eliminated. The Mitomaster website has defined a mitochondrial haplogroup. Variants presenting a frequency in the haplogroup 50.5% or if they were described in cancers were retained. Any sequencing errors were checked on IGV or were automatically filtered by a bioinformatics program. For haplogroups whose number is <100, the frequency of the variant was checked in gnomAD v3 and retained if <0.5% or if described in cancers (FIG. 1).

### Development of Mitoscores:

From the survival curves carried out individually by gene of the mitochondrial genome in the 64 patients in the study who received an induction chemotherapy, certain genes were determined to have a «good prognosis» (ND2/ND3/ND4/ATP8/CYTB/ND5/ATP6) or «poor prognosis» (COX1/COX2/COX3/12S/ND1).

Despite a good separation of the survival curves, no significant differences were observed in the univariate analysis of these genes taken individually.

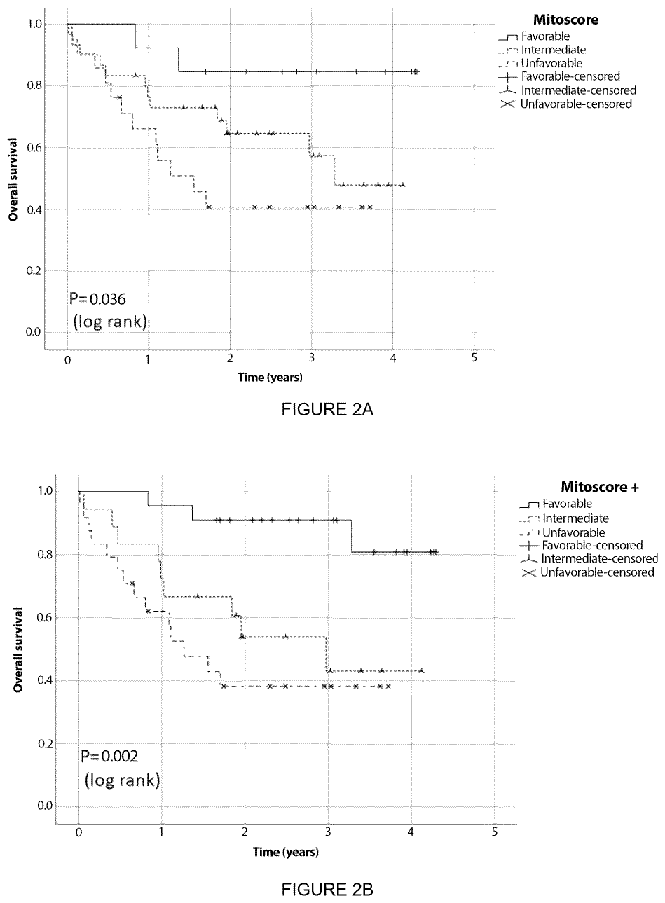

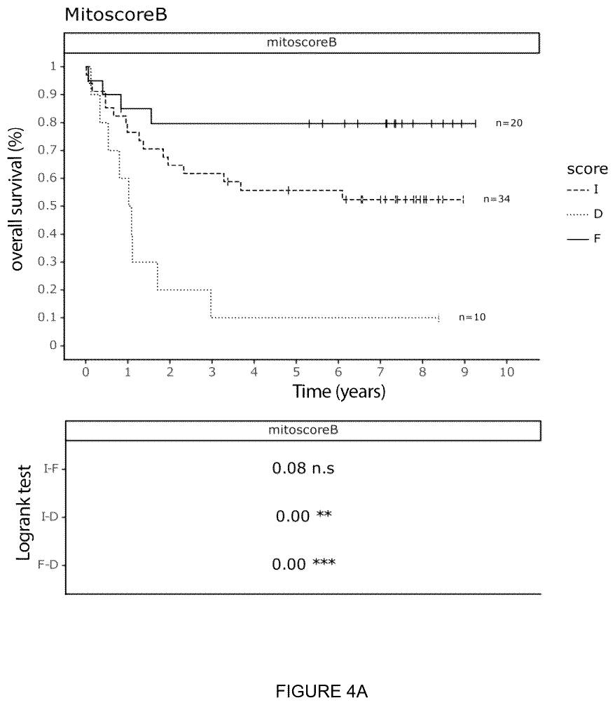

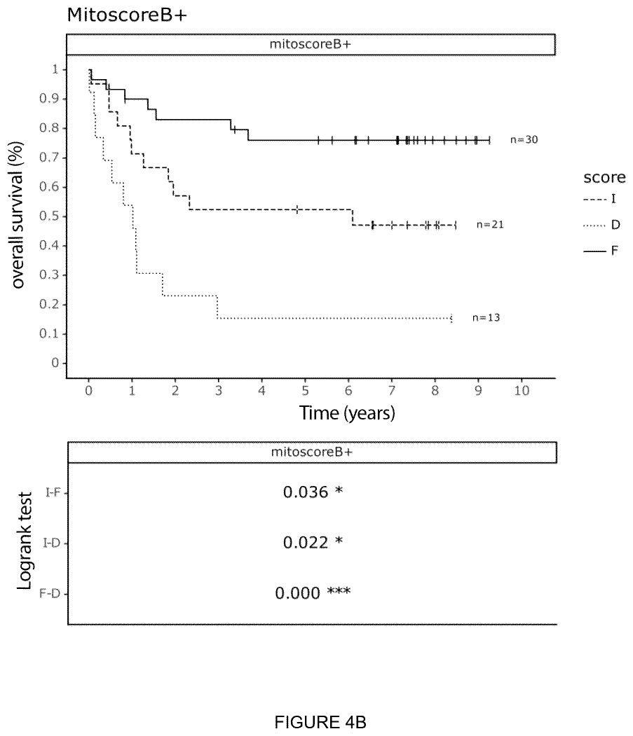

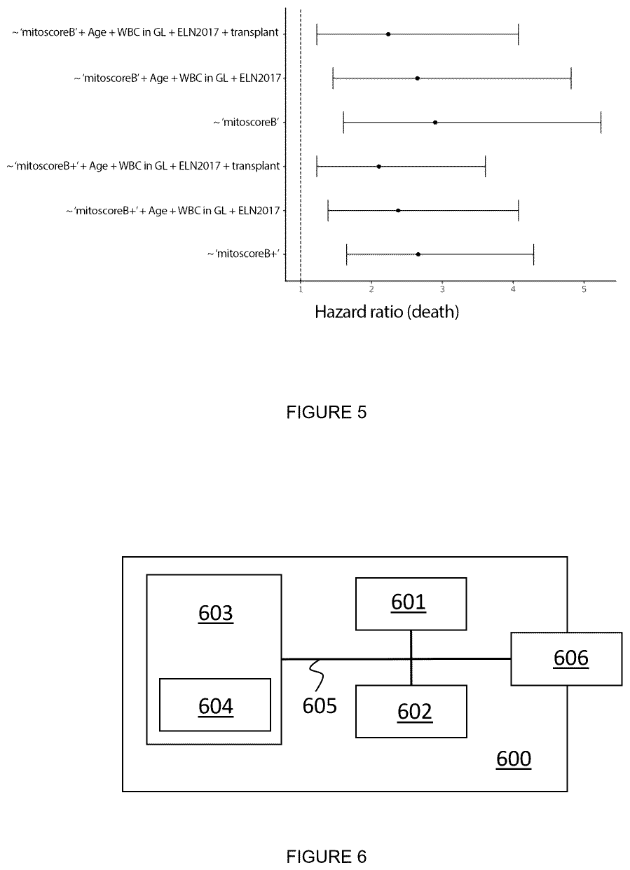

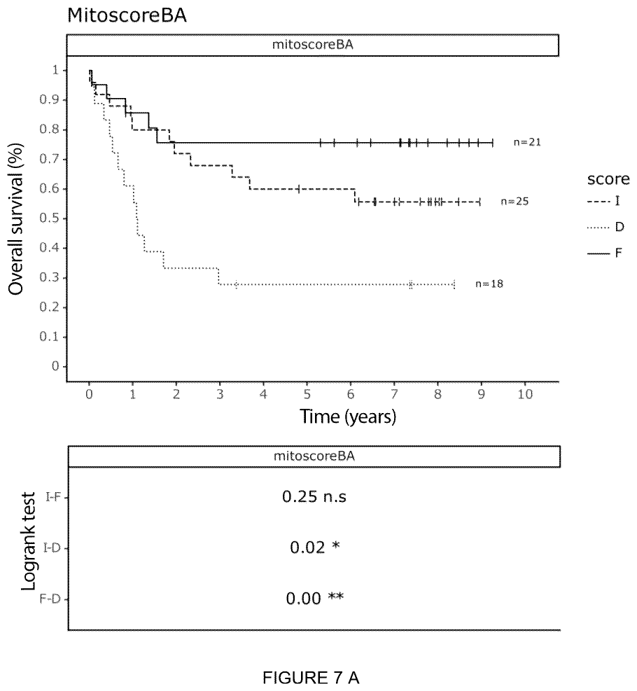

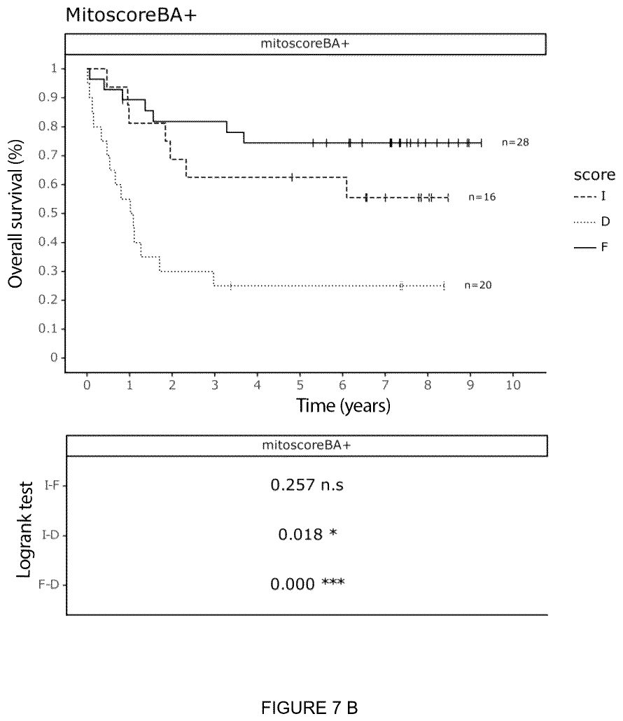

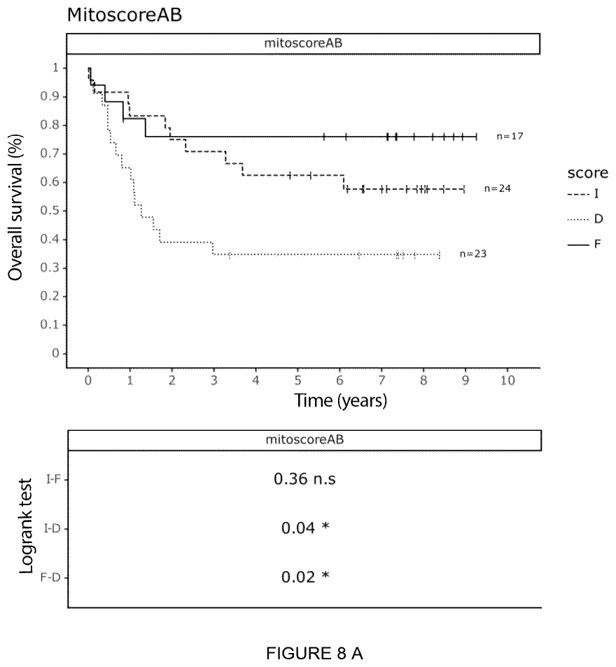

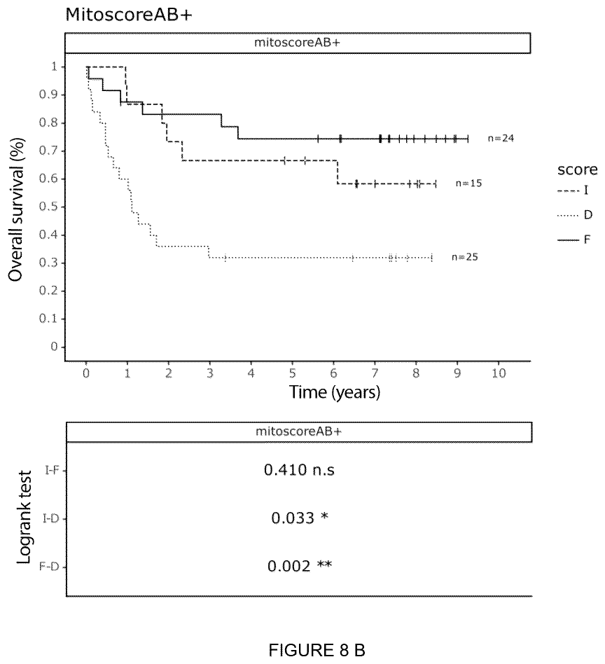

Therefore, «Mitoscores» were created, stratifying the patients into 3 groups of respective favorable, intermediate, unfavorable prognosis (FIG. 2A/FIG. 4A).

This prognostic classification based on “Mitoscore” is independent of the usual prognostic factors for AML (age, WBC/I, ELN 2017, bone marrow transplant).

However, 30% of the patients without any mutation in all the genes of Mitoscore, i.e. patients of the subgroup called Mitonaïve A, require a reclassification. Therefore, Mitoscore+ was created by combining the prognostic classification of the ELN 2017 with Mitoscore in Mitonaïve patients (FIG. 2B). This allows a reduction in the use of the prognostic classification of the ELN 2017 in the range of 70%, 40% of the patients without any mutation in all of the genes of Mitoscore B, i.e. patients of the subgroup called Mitonaïve B, require a reclassification. Therefore, Mitoscore B+ was created by combining the prognostic classification of the ELN 2017 with Mitoscore in Mitonaïve patients (FIG. 4B). This allows a reduction in the use of the prognostic classification of the ELN 2017 in the range of 60%.

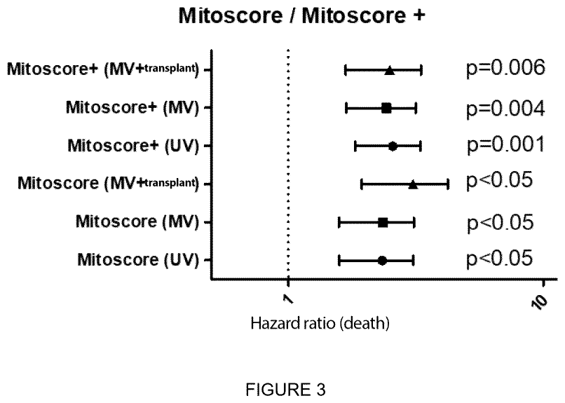

The interest of Mitoscore and Mitoscore+ was confirmed with a Cox model in univariate analysis (UV) independently of the usual prognostic factors of AML and in multivariate analysis (MV) (FIG. 3). The covariates used in the MV analysis were: age, number of white blood cells (WBC) circulating in G/l, ELN 2017 classification, and/or bone marrow transplant. Likewise, Mitoscores B/B+ are significant in multivariate analysis (in g/).

Additionally, Mitoscore was functionally characterized by comparing to ROS emission data. Interestingly, the leukemic cells of the patients in the “unfavorable” Mitoscore subgroup emit significantly less ROS under antimycin/rotenone stimulation conditions (targeting mitochondrial ROS) than those of the «favorable» and «intermediate» Mitoscore with the mutations COX1/COX2/COX3/12S and ND2/ND3/ATP8/CYTB/ND4.

Within mitochondria, the complexes I and III of the respiratory chain are the main sources of reactive oxygen species emission. The genes involved in the «favorable» Mitoscore affect subunits of the complexes I (ND2/ND3/ND4/ND5) and III (CYTB) as well as ATP synthase (ATP8/ATP6). The ROS data therefore support the hypothesis of “favorable” Mitoscore functioning where the mutations affect the emission of ROS, particularly under stimulation conditions such as induced by the chemotherapy. Thus, the strong emission of ROS triggers the apoptosis threshold and leads to cell death of the leukemia cells.

In the «unfavorable» Mitoscore subgroup, fewer ROS are emitted. Indeed, the complex IV of the respiratory chain encoded by the COX1/COX2/COX3 genes is not a site of superoxide ion emission. Furthermore, the Mitochondrial-derived peptide MOTS-c encoded by the 12S gene plays a role in the metabolic adaptation to stress.

Mitoscore+ was also functionally characterized in the same way as for Mitoscore. In the subgroup of patients called Mitonaïve, the patients whose leukemia cells are capable of producing more ROS in the presence of antimycin/rotenone and DPI (diphenyleneiodonium) have significantly a better overall survival compared to the patients whose leukemia cells produce less ROS.

### Definition of Mitoscore and Mitoscore B:

Mitoscore made it possible to stratify the 64 AML patients into 3 prognosis groups in terms of overall survival based on the combinations of the following variants:

A «favorable» Mitoscore corresponds to the presence of at least one mutation in one of the ND2/ND3/ATP8/CYTB/ND4 genes or of at least one mutation in one of the ND2/ND3/ATP8/CYTB genes, and the absence of mutations in the following COX1/COX2/COX3/12S genes.

An «unfavorable» Mitoscore corresponds to the presence of at least one mutation in one of the COX1/COX2/COX3/12S genes, and the absence of mutations in the following ND2/ND3/ATP8/CYTB/ND4 genes.

An «Intermediate» Mitoscore corresponds to:
either the presence of at least one mutation in one of the ND2/ND3/ATP8/CYTB/ND4 genes, and of at least one mutation in one of the COX1/COX2/COX3/12S genes. This is the subgroup of patients called «mutated intermediate»;
or the absence of mutations in the ND2/ND3/ATP8/CYTB/ND4/COX1/COX2/COX3/12S genes. This is the subgroup of patients called «Mitonaïve A»), corresponding to approximately 30% of the AML patients.

TABLE 2

| Mitoscore | Variants retained according to Work Flow |
|---|---|
| Favorable | presence of at least one variant retained in the |
| | ND2/ND3/ATP8/CYTB/ND4 genes |
| | and |
| | no variant retained in the COX1/ |
| | COX2/COX3/12S genes |
| Intermediate | Either subgroup called mutated intermediate: |
| | presence of at least one variant retained in the |
| | COX1/COX2/COX3/12S genes |
| | and |
| | presence of at least one variant retained in the |
| | ND2/ND3/ATP8/CYTB/ND4 genes |
| | Or subgroup called Mitonaïve A: |
| | no variant retained in the |
| | COX1/COX2/COX3/12S/ND2/ND3/ |
| | ATP8/CYTB/ND4 genes |
| Unfavorable | presence of at least one variant retained in the |
| | COX1/COX2/COX3/12S genes |
| | and |
| | no variant retained in the ND2/ND3/ |
| | ATP8/CYTB/ND4 genes |

Mitoscore B also made it possible to stratify the 64 AML patients into 3 prognosis groups in terms of overall survival based on the combinations of the following variants:

A «favorable» Mitoscore also corresponds to the presence of at least one mutation in one of the ND2/ND5/ATP6/CYTB/ND4 genes, and the absence of mutations in the following ND1/COX3/12S genes.

An «unfavorable» Mitoscore also corresponds to the presence of at least one mutation in one of the ND1/COX3/12S genes, and the absence of mutations in the following ND2/ND5/ATP6/CYTB/ND4 genes.

An «intermediate» Mitoscore also corresponds to:
either the presence of at least one mutation in one of the ND2/ND5/ATP6/CYTB/ND4 genes, and of at least one mutation in one of the ND1/COX3/12S genes. This is the subgroup of patients called «mutated intermediate»;
or the absence of mutations in the ND2/ND5/ATP6/CYTB/ND4/ND1/COX3/12S genes. This is the subgroup of patients called «Mitonaïve B»), corresponding to approximately 40% of the AML patients.

TABLE 3

| Mitoscore | Variants retained according to Work Flow |
|---|---|
| Favorable | presence of at least one variant retained in the |
| | ND2/ND5/ATP6/CYTB/ND4 genes |
| | and |
| | no variant retained in the ND1/COX3/12S genes |
| Intermediate | Either subgroup called mutated intermediate: |
| | presence of at least one variant |
| | retained in the ND1/COX3/12S |
| | genes |
| | and |
| | presence of at least one variant retained in the |
| | ND2/ND5/ATP6/CYTB/ND4 genes |
| | Or subgroup called Mitonaïve B: |
| | no variant retained in the |
| | ND1/COX3/12S/ND2/ND5/ATP6/CYTB/ND4 genes |
| Unfavorable | presence of at least one variant |
| | retained in the ND1/COX3/12S |
| | genes and no variant retained in the |
| | ND2/ND5/ATP6/CYTB/ND4 genes |

### Definition of Mitoscore+ and Mitoscore B+:

In the subgroup of patients called Mitonaïve A of the «intermediate» Mitoscore, the stratification of ELN was applied to determine Mitoscore+ which made it possible to reclassify these 30% of AML patients into 3 prognosis groups in terms of overall survival based on the combinations of the following variant:

A «favorable» Mitoscore+ corresponds to:
either the presence of at least one mutation in one of the ND2/ND3/ATP8/CYTB/ND4 genes or of at least one mutation in one of the ND2/ND3/ATP8/CYTB genes, and the absence of mutations in the following COX1/COX2/COX3/12S genes;
or the Mitonaïve A patients classified “favorable” according to the ELN score.

An “unfavorable” Mitoscore+ corresponds to:
either the presence of at least one mutation in one of the COX1/COX2/COX3/12S genes, and the absence of mutations in the following ND2/ND3/ATP8/CYTB/ND4 genes;
or the Mitonaïve A patients classified “unfavorable” according to the ELN score.

The «intermediate» Mitoscore+ corresponds to:
either the presence of at least one mutation in one of the ND2/ND3/ATP8/CYTB/ND4 genes, and of at least one mutation in one of the COX1/COX2/COX3/12S genes;
or the Mitonaïve A patients classified “intermediate” according to the ELN score.

TABLE 4

| Mitoscore+ | Variants retained according to Work Flow |
|---|---|
| Favorable | Either: |
| | presence of at least one variant retained in the |
| | ND2/ND3/ATP8/CYTB/ND4 genes and |
| | no variant retained in |
| | the COX1/COX2/COX3/12S genes |
| | Or: |
| | Mitonaïve A subgroup |
| | (see description Mitoscore) Favorable |
| | according to the ELN |
| Intermediate | Either: |
| | presence of at least one variant retained in the |
| | COX1/COX2/COX3/12S genes and presence of at least one |
| | variant retained in the ND2/ND3/ATP8/CYTB/ND4 genes |
| | Or: |
| | Mitonaïve A subgroup |
| | (see description Mitoscore) Intermediate |
| | according to the ELN |
| Unfavorable | Either: |
| | presence of at least one variant retained in the |
| | COX1/COX2/COX3/12S genes and no |
| | variant retained in the |
| | ND2/ND3/ATP8/CYTB/ND4 genes |
| | Or: |
| | Mitonaïve A subgroup |
| | (see description Mitoscore) Unfavorable |
| | according to the ELN |

In the subgroup of patients called Mitonaïve B of the “intermediate” Mitoscore, the stratification of the ELN was also applied to determine Mitoscore B+ which made it possible to reclassify these 40% of AML patients into 3 prognosis groups in terms of overall survival based on the combinations of the following variant:

A «favorable» Mitoscore B+ corresponds to:
either the presence of at least one mutation in one of the ND2/ND5/ATP6/CYTB/ND4 genes, and the absence of mutations in the following ND1/COX3/12S genes;
or the Mitonaïve B patients classified “favorable” according to the ELN score.

An «unfavorable» Mitoscore B+ corresponds to:
either the presence of at least one mutation in one of the ND1/COX3/12S genes, and the absence of mutations in the following ND2/ND5/ATP6/CYTB/ND4 genes;
or the Mitonaïve B patients classified “unfavorable” according to the ELN score.

An «intermediate» Mitoscore B+ corresponds to:
either the presence of at least one mutation in one of the ND2/ND5/ATP6/CYTB/ND4 genes, and of at least one mutation in one of the ND1/COX3/12S genes;
or the Mitonaïve B patients classified “intermediate” according to the ELN score.

TABLE 5

| Mitoscore B+ | Variants retained according to Work Flow |
|---|---|
| Favorable | Either: |
| | presence of at least one variant retained in the |
| | ND2/ND5/ATP6/CYTB/ND4 genes |
| | and no variant retained in |
| | the ND1/COX3/12S genes |
| | Or: |
| | Mitonaïve B |
| | subgroup (see description Mitoscore B) |
| | Favorable according to the ELN |
| Intermediate | Either: |
| | presence of at least one variant retained in the |
| | ND1/COX3/12S genes and presence |
| | of at least one variant |
| | retained in the ND2/ND5/ATP6/CYTB/ND4 genes |
| | Or: |
| | Mitonaïve B |
| | subgroup (see description Mitoscore B) |
| | Intermediate according to the ELN |
| Unfavorable | Either: |
| | presence of at least one variant retained in the |
| | ND1/COX3/12S genes and no variant retained in the |
| | ND2/ND5/ATP6/CYTB/ND4 genes |
| | Or: |
| | Mitonaïve B |
| | subgroup (see description Mitoscore B) |
| | Unfavorable according to the ELN |

### Definition of Mitoscore B/A:

Mitoscore B/A also made it possible to stratify the 64 AML patients into 3 prognosis groups in terms of overall survival based on the combinations of the following variants:

A «favorable» Mitoscore B/A corresponds to:
either the presence of at least one mutation in the ND2/ND5/ATP6/CYTB/ND4 genes, and the absence of a variant in the ND1/COX3/12S genes;
or the absence of a variant in the ND1/COX3/12S/ND2/ND5/ATP6/CYTB/ND4/COX1/COX2 genes, and the presence of at least one mutation in the ATP8/ND3 genes.

An «unfavorable» Mitoscore B/A corresponds to:
either the presence of at least one mutation in the ND1/COX3/12S genes, and the absence of a variant in the ND2/ND5/ATP6/CYTB/ND4 genes;
or the absence of a variant in the ND1/COX3/12S/ND2/ND5/ATP6/CYTB/ND4/ATP8/ND3 genes, and the presence of at least one mutation in the COX1/COX2 genes.

An “intermediate” Mitoscore B/A corresponds to:
either the presence of at least one mutation in the ND1/OX3/12S genes, and the presence of at least one mutation in the ND2/ND5/ATP6/CYTB/ND4 genes;
or the absence of a variant in the ND1/COX3/12S/ND2/ND5/ATP6/CYTB/ND4 genes, and the presence of at least one mutation in the COX1 or COX2 genes, and the presence of at least one mutation in the ATP8 and ND3 genes;
or the absence of a variant in the ND1/NOX3/12S/ND2/ND5/ATP6/CYTB/ND4/COX1/COX2/ATP8/ND3 genes.

TABLE 6

| Mitoscore B/A | Variants retained according to Work Flow |
|---|---|
| Favorable | Either: |
| | presence of at least one mutation retained |
| | in the ND2/ND5/ATP6/CYTB/ND4 genes |
| | and no variant retained |
| | in the ND1/COX3/12S genes |
| | Or: |
| | no variant retained in the |
| | ND1/COX3/12S/ND2/ND5/ |
| | ATP6/CYTB/ND4/COX1/COX2 |
| | and presence of at least one variant |
| | retained in the ATP8/ND3 genes |
| Intermediate | Either: |
| | presence of at least one variant retained |
| | in the ND1/COX3/12S genes and presence |
| | of at least one variant retained |
| | in the ND2/ND5/ATP6/CYTB/ND4 genes |
| | Or: |
| | no variant retained in the |
| | ND1/COX3/12S/ND2/ND5/ATP6/CYTB/ND4 genes and |
| | presence of at least one variant retained in the COX1 or |
| | COX2 genes and presence of at least one variant retained |
| | in the ATP8 and ND3 genes |
| | Or: |
| | no variant retained in the |
| | ND1/COX3/12S/ND2/ND5/ATP6/CYTB/ |
| | ND4/COX1/COX2/ATP8/ND3 genes |
| Unfavorable | Either: |
| | presence of at least one variant retained in the |
| | ND1/COX3/12S genes and no variant retained in the |
| | ND2/ND5/ATP6/CYTB/ND4 genes |
| | Or: |
| | no variant retained in the |
| | ND1/COX3/12S/ND2/ND5/ |
| | ATP6/CYTB/ND4/ATP8/ND3 |
| | genes and presence of at least |
| | one variant retained in the |
| | COX1/COX2 genes |

### Definition of Mitoscore A/B:

Mitoscore A/B made it possible to stratify the 64 AML patients into 3 prognosis groups in terms of overall survival based on the combinations of the following variants:

A «favorable» Mitoscore A/B corresponds to:
either the presence of at least one mutation in the ND2/ND3/ATP8/CYTB/ND4 genes, and the absence of a variant in the COX1/COX2/COX3/12S genes;
or the absence of a variant in the COX1/COX2/COX3/12S/ND2/ND3/ATP8/CYTB/ND4/ND1 genes, and the presence of at least one mutation in the ATP6/ND5 genes.

An «unfavorable» Mitoscore A/B corresponds to:
either the presence of at least one mutation in the COX1/COX2/COX3/12S genes, and the absence of a variant in the ND2/ND3/ATP8/CYTB/ND4 genes;
or the absence of a variant in the COX1/COX2/COX3/12S/ND2/ND3/ATP8/CYTB/ND4/ATP6/ND5 genes, and the presence of at least one mutation in the ND1 gene.

An “intermediate” Mitoscore A/B corresponds to:
either the presence of at least one mutation in the COX1/COX2/COX3/12S genes, and the presence of at least one mutation in the ND2/ND3/ATP8/CYTB/ND4 genes;
or the absence of a variant in the COX1/COX2/COX3/12S/ND2/ND3/ATP8/CYTB/ND4 genes, and the presence of at least one mutation in the ATP6/ND5 genes and of at least one mutation in the ND1 gene;
or the absence of a variant in the COX1/COX2/COX3/12S/ND2/ND3/ATP8/CYTB/ND4/ATP6/ND5/ND1 genes.

TABLE 7

| Mitoscore A/B | Variants retained according to Work Flow |
|---|---|
| Favorable | Either: |
| | presence of at least one variant retained in the |
| | ND2/ND3/ATP8/CYTB/ND4 genes |
| | and no variant retained |
| | in the COX1/COX2/COX3/12S genes |
| | Or: |
| | no variant retained in the |
| | COX1/COX2/COX3/12S/ND2/ |
| | ND3/ATP8/CYTB/ND4/ND1 |
| | genes and presence of at least one |
| | variant retained in the ATP6/ND5 genes |
| Intermediate | Either: |
| | presence of at least one variant retained |
| | in the COX1/COX2/COX3/12S genes and |
| | presence of at least one variant |
| | retained in the ND2/ND3/ATP8/CYTB/ND4 genes |
| | Or: |
| | no variant retained in the |
| | COX1/COX2/COX3/12S/ND2/ |
| | ND3/ATP8/CYTB/ND4 genes |
| | and presence of at least |
| | one variant retained in the |
| | ATP6/ND5 genes and at least one |
| | variant retained in the ND1 gene |
| | Or: |
| | no variant retained in the |
| | COX1/COX2/COX3/12S/ND2/ND3/ |
| | ATP8/CYTB/ND4/ATP6/ND5/ND1 genes |
| Unfavorable | Either: |
| | presence of at least one variant retained |
| | in the COX1/COX2/COX3/12S genes |
| | and no variant retained in |
| | the ND2/ND3/ATP8/CYTB/ND4 genes |
| | Or: |
| | no variant retained in the |
| | COX1/COX2/COX3/12S/ND2/ND3/ |
| | ATP8/CYTB/ND4/ATP6/ND5 |
| | genes and presence of at least one |
| | variant retained in the ND1 gene |

### Definition of Mitoscore B/A+:

Mitoscore B/A+ also made it possible to stratify the 64 AML patients into 3 prognosis groups in terms of overall survival based on the combinations of the following variants:

A «favorable» Mitoscore B/A+ corresponds to:
either the presence of at least one variant in the ND2/ND5/ATP6/CYTB/ND4 genes, and the absence of a variant in the ND1/COX3/12S genes;
or the absence of a variant in the ND1/COX3/12S/ND2/ND5/ATP6/CYTB/ND4/COX1/COX2 genes, and the presence of at least one variant in the ATP8/ND3 genes;
or the absence of a variant in the ND1/COX3/12S/ND2/ND5/ATP6/CYTB/ND4/COX1/COX2/ATP8/ND3 genes, and favorable ELN.

An «unfavorable» Mitoscore B/A+ corresponds to:
either the presence of at least one variant in the ND1/OX3/12S genes, and the absence of a variant in the ND2/ND5/ATP6/YTB/ND4 genes;
or the absence of a variant in the ND1/COX3/12S/ND2/ND5/ATP6/CYTB/ND4/ATP8/ND3 genes, and the presence of at least one variant in the COX1/COX2 genes;
or the absence of a variant in the ND1/NOX3/12S/ND2/ND5/ATP6//YTB/ND4/AOX1/COX2/ATP8/ND3 genes, and unfavorable ELN.

An “intermediate” Mitoscore B/A+ corresponds to:
either the presence of at least one variant in the ND1/COX3/12S genes, and the presence of at least one variant in the ND2/ND5/ATP6/YTB/ND4 genes;
or the absence of a variant in the ND1/NOX3/12S/ND2/ND5/ATP6//YTB/ND4 genes, and the presence of at least one variant retained in the COX1/COX2 genes and the presence of at least one variant in the ATP8/ND3 genes;
or the absence of a variant in the ND1/NOX3/12S/ND2/ND5/ATP6/CYTB/ND4/OX1/COX2/ATP8/ND3 genes, and intermediate ELN.

TABLE 8

| Mitoscore B/A+ | Variants retained according to Work Flow |
|---|---|
| Favorable | Either: |
| | presence of at least one variant retained in the |
| | ND2/ND5/ATP6/CYTB/ND4 genes and no |
| | variant retained in the ND1/COX3/12S genes |
| | Or: |
| | no variant retained in the |
| | ND1/COX3/12S/ND2/ND5/ |
| | ATP6/CYTB/ND4/COX1/COX2 |
| | genes and presence of at least one variant |
| | retained in the ATP8/ND3 genes |
| | Or: |
| | no variant retained in the |
| | ND1/COX3/12S/ND2/ND5/ATP6/ |
| | CYTB/ND4/COX1/COX2/ATP8/ND3 |
| | genes and favorable ELN |
| Intermediate | Either: |
| | presence of at least one variant retained in the |
| | ND1/COX3/12S genes and presence |
| | of at least one variant retained |
| | in the ND2/ND5/ATP6/CYTB/ND4 genes |
| | Or: |
| | no variant retained in the |
| | ND1/COX3/12S/ND2/ND5/ |
| | ATP6/CYTB/ND4 genes and |
| | presence of at least one variant |
| | retained in the COX1/COX2 genes and |
| | presence of at least one variant retained |
| | in the ATP8/ND3 genes |
| | Or: |
| | no variant retained in the |
| | ND1/COX3/12S/ND2/ND5/ATP6/ |
| | CYTB/ND4/COX1/COX2/ATP8/ND3 |
| | genes and intermediate ELN |
| Unfavorable | Either: |
| | presence of at least one variant retained in the |
| | ND1/COX3/12S genes and no variant retained |
| | in the ND2/ND5/ATP6/CYTB/ND4 genes |
| | Or: |
| | no variant retained in the |
| | ND1/COX3/12S/ND2/ND5/ |
| | ATP6/CYTB/ND4/ATP8/ND3 |
| | genes and presence of at least one variant |
| | retained in the COX1/COX2 genes |
| | Or: |
| | no variant retained in the |
| | ND1/COX3/12S/ND2/ND5/ATP6/ |
| | CYTB/ND4/COX1/COX2/ATP8/ND3 |
| | genes and unfavorable ELN |

### Definition of Mitoscore A/B+:

The Mitoscore A/B+ made it possible to stratify the 64 AML patients into 3 prognosis groups in terms of overall survival based on the combinations of the following variants:

A «favorable» Mitoscore A/B+ corresponds to:
either the presence of at least one variant in the ND2/ND3/ATP8/CYTB/ND4 genes, and the absence of a variant in the COX1/COX2/COX3/12S genes;
or the absence of a variant in the COX1/COX2/COX3/12S/ND2/ND3/ATP8/CYTB/ND4/ND1 genes, and the presence of at least one variant in the ATP6/ND5 genes;
or the absence of a variant in the COX1/COX2/COX3/12S/ND2/ND3/ATP8/CYTB/ND4/ATP6/ND5/ND1 genes, and favorable ELN.

An «unfavorable» Mitoscore A/B+ corresponds to:
either the presence of at least one variant in the COX1/COX2/COX3/12S genes, and the absence of a variant in the ND2/ND3/ATP8/CYTB/ND4 genes;
or the absence of a variant in the COX1/COX2/COX3/12S/ND2/ND3/ATP8/CYTB/ND4/ATP6/ND5 genes, and the presence of at least one variant in the ND1 gene;
or the absence of a variant in the COX1/COX2/COX3/12S/ND2/ND3/ATP8/CYTB/ND4/ATP6/ND5/ND1 genes, and unfavorable ELN.

An “intermediate” Mitoscore A/B+ corresponds to:
either the presence of at least one variant in the COX1/COX2/COX3/12S genes, and the presence of at least one variant in the ND2/ND3/ATP8/CYTB/ND4 genes;
or the absence of a variant in the COX1/COX2/COX3/12S/ND2/ND3/ATP82/YTB/ND4 genes, and the presence of at least one variant in the ATP6ND5 genes and of at least one variant in the ND1 gene;
or the absence of a variant in the COX1/COX2/COX3/12S/ATP8/CYTB/ND4/ATP6/ND5/ND1 genes, and intermediate ELN.

TABLE 9

| Mitoscore A/B+ | Variants retained according to Work Flow |
|---|---|
| Favorable | Either: |
| | presence of at least one variant retained |
| | in the ND2/ND3/ATP8/CYTB/ND4 genes |
| | and no variant retained in the |
| | COX1/COX2/COX3/12S genes |
| | Or: |
| | no variant retained in the |
| | COX1/COX2/COX3/12S/ND2/ |
| | ND3/ATP8/CYTB/ND4/ND1 |
| | genes and presence of at least |
| | one variant retained in the |
| | ATP6/ND5 genes |
| | Or: |
| | no variant retained in the |
| | COX1/COX2/COX3/12S/ND2/ND3/ |
| | ATP8/CYTB/ND4/ATP6/ND5/ND1 |
| | genes and favorable ELN |
| Intermediate | Either: |
| | presence of at least one variant retained |
| | in the COX1/COX2/COX3/12S genes and |
| | presence of at least one |
| | variant retained in the ND2/ND3/ |
| | ATP8/CYTB/ND4 genes |
| | Or: |
| | no variant retained in the |
| | COX1/COX2/COX3/12S/ND2/ |
| | ND3/ATP8/CYTB/ND4 genes |
| | and presence of at least one variant retained |
| | in the ATP6/ND5 genes and at least one variant |
| | retained in the ND1 gene |
| | Or: |
| | no variant retained in the |
| | COX1/COX2/COX3/12S/ND2/ND3/ |
| | ATP8/CYTB/ND4/ATP6/ND5/ND1 |
| | genes and intermediate ELN |
| Unfavorable | Either: |
| | presence of at least one variant retained in the |
| | COX1/COX2/COX3/12S genes and no variant retained |
| | in the ND2/ND3/ATP8/CYTB/ND4 genes |
| | Or: |
| | no variant retained in the |
| | COX1/COX2/COX3/12S/ND2/ND3/ |
| | ATP8/CYTB/ND4/ATP6/ND5 |
| | genes and presence of at least one variant |
| | retained in the ND1 genes |
| | Or: |
| | no variant retained in the |
| | COX1/COX2/COX3/12S/ND2/ND3/ |
| | ATP8/CYTB/ND4/ATP6/ND5/ND1 |
| | genes and unfavorable ELN |

The different Mitoscores according to the invention may thus show their effectiveness in order to predict the response to treatments for chemosensitive cancer, for example using venetoclax. Thus, they make it possible to define the sensitivity to a treatment (e.g. to venetoclax) in the acute myeloid leukemia indication as well as in other indications using the treatment.

### Definition of Mitoscores as a Companion Test:

In intermediate and unfavorable patients with Mitoscores, Mitoscores according to the invention may make it possible to modify the management in combination with the chemotherapy using drugs capable of:
targeting the communication between the mitochondria and the leukemia cell nucleus by administering the MOTS-c peptide in intermediate or unfavorable patients with a mutation in the 12S gene
targeting the complex IV of the mitochondria with for example quercetin or other drugs in intermediate or unfavorable patients with a mutation in one of the COX1, COX2, COX3 genes
targeting the complexes I and III of the mitochondrial respiratory chain in intermediate or unfavorable patients via the administration of drugs targeting the mitochondria, for example olaparib, mubritinib, trimetazidine dihydrochloride, myxothiazol . . . .

Thus Mitoscores of the invention may serve as a companion test to improve the management of patients and define possible indications for adding complementary therapies/products targeting the mitochondria.

### Computer Program for Implementing Mitochondrial Genotypical Scores

A computer program was created for the implementation of the above scores in order to be able to evaluate the effectiveness of a therapeutic treatment, for example venetoclax, vis-à-vis a chemosensitive cancer, in particular AML. Said program analyzes the variants from the source data of the sequencer used, filters them according to quality criteria and the location of the variants in the coding areas, the heteroplasmy rate and silent mutations. The program then applies Mitoscores (Mitoscore/Mitoscore+, Mitoscore B/Mitoscore B+, Mitoscore B/A, Mitoscore B/A+, Mitoscore A/B, Mitoscore A/B+).

FIG. 6 is a block diagram of a device 600 configured to implement the steps of a method for establishing a survival prognosis in a patient suffering from chemosensitive cancer, as described above. The device comprises a processor 601, a volatile memory 602, a non-volatile memory 603, the latter including instructions 604 which, when executed by the processor 601, lead the device 600 to implement the steps mentioned above to establish a survival prognosis in a patient suffering from chemosensitive cancer. The different components are connected by a communication bus 605. The device may include other components or interfaces 606 to other components, depending on the implementation, in particular a user interface. The processor 601 may take any suitable form—one or more microprocessors, one or more microcontrollers, or a combination thereof. The volatile memory 602 is used for temporary data storage. The non-volatile memory 603 may comprise a hard drive, a static memory, or other form of long-term storage. The non-volatile memory stores the implementation instructions, it may also store an operating system and/or applications. The device 600 is for example a computer into which a computer program is loaded or downloaded which includes the instructions 604.

The different Mitoscores according to the invention may thus show their effectiveness in order to predict the response to treatments using venetoclax. For example, they make it possible to define the sensitivity to venetoclax in the acute myeloid leukemia indication as well as in other indications using the treatment.

### LIST OF REFERENCES

1. Dohner et al., Blood, 129(4):424-447, 2017
2. Herold et al, Leukemia, 34(12):3161-3172, 2020
3. Dohner et al., Blood, 140(12): 1345-1377, 2022
4. Farge et al., Cancer discov., 7(7):716-735, 2017
5. Mondet et al, Hematologica, 104(9):e393-e397, 2019
6. Bosc et al., Nat. Cancer., 2(11): 1204-1223, 2021
7. Neuzil et al., Mitochondrion, 13(3): 199-208, 2013
8. Damm et al., Leukemia, 26: 289-295, 2012
9. Chun et al., Chin. J. Hematol., 35(8): 708-712, 2014
10. Wu et al., Scientific reports, 8: 13301, 2018
11. Caudron-Herger and Diederichs, Biology, 15: 62-69, 2018
12. Zeng et al., Scientific reports, 8: 3623, 2018
13. Silkjaer et al., Eur. J. Haematol., 90(5): 385-396, 2013
14. Silkjaer et al., Eur. J. Haematol., 91(4): 295-303, 2013

1. An In vitro method for establishing a survival prognosis in a patient suffering from chemosensitive cancer, said method comprising the following steps of:
detecting in a biological sample of said patient, the presence and/or absence of at least one mutation on one of the following nine genes of the mitochondrial genome: ND1, ND2, ND3, ND4, ND5, CYTB, ATP8, ATP6, COX1, COX2, COX3 and 12S;
comparing detection results compared to a reference biological sample or a reference sequence or a reference haplogroup/haplotype;
establishing the survival prognosis of said patient.

2. The method according to claim 1, wherein:
the presence of at least one mutation in one of the ND2, ND3, ATP8, CYTB, ND4 genes and the absence of mutations in the COX1, COX2, COX3, 12S genes, is the indication of a favorable survival prognosis;
the presence of at least one mutation in one of the COX1, COX2, COX3, 12S genes and the absence of mutations in the ND2, ND3, ATP8, CYTB, ND4 genes, is the indication of an unfavorable survival prognosis; and
either the absence of mutations in the ND2, ND3, ATP8, CYTB, ND4, COX1, COX2, COX3, 12S genes (Mitonaïve A patients), or the presence of at least one mutation in one of the ND2, ND3, ATP8, CYTB, ND4 genes and of at least one mutation in one of the COX1, COX2, COX, 12S genes, is the indication of an intermediate survival prognosis.

3. The method according to claim 1, wherein:
the presence of at least one mutation in one of the ND2/ND5/ATP6/CYTB/ND4 genes, and the absence of mutations in the following ND1/COX3/12S genes, is the indication of a favorable survival prognosis;
the presence of at least one mutation in one of the ND1/COX3/12S genes, and the absence of mutations in the following ND2/ND5/ATP6/CYTB/ND4 genes, is the indication of an unfavorable survival prognosis; and
either the absence of mutations in the ND2/ND5/ATP6/CYTB/ND4/ND1/COX3/12S genes (Mitonaïve B patients), or the presence of at least one mutation in one of the ND2/ND5/ATP6/CYTB/ND4 genes and of at least one mutation in one of the ND1/COX3/12S genes, is the indication of an intermediate survival prognosis.

4. The method according to claim 2, wherein the method further comprises determining the ELN prognostic score in the subgroup of Mitonaïve A or Mitonaïve B patients.

5. The method according to claim 4, wherein:
either the presence of at least one mutation in one of the ND2/ND3/ATP8/CYTB/ND4 genes and the absence of mutations in the COX1/COX2/COX3/12S genes, or the absence of mutations in the ND2, ND3, ATP8, CYTB, ND4, COX1, COX2, COX3, 12S genes and a “favorable” classification according to the ELN score, is the indication of a favorable survival prognosis;
either the presence of at least one mutation in one of the COX1/COX2/COX3/12S genes and the absence of mutations in the ND2/ND3/ATP8/CYTB/ND4 genes, or the absence of mutations in the ND2, ND3, ATP8, CYTB, ND4, COX1, COX2, COX3, 12S genes and an “unfavorable” classification according to the ELN score, is the indication of an unfavorable survival prognosis;
either the presence of at least one mutation in one of the ND2/ND3/ATP8/CYTB/ND4 genes and at least one mutation in one of the COX1/COX2/COX3/12S genes, or the absence of mutations in the ND2, ND3, ATP8, CYTB, ND4, COX1, COX2, COX3, 12S genes and an «intermediate» classification according to the ELN score, is the indication of an intermediate survival prognosis.

6. The method according to claim 4, wherein:
either the presence of at least one mutation in one of the ND2/ND5/ATP6/CYTB/ND4 genes and the absence of mutations in the following ND1/COX3/12S genes, or the absence of mutations in the ND2, ND5, ATP6, CYTB, ND4, ND1, COX3, 12S genes and a “favorable” classification according to the ELN score, is the indication of a favorable survival prognosis;
either the presence of at least one mutation in one of the ND1/COX3/12S genes and the absence of mutations in the following ND2/ND5/ATP6/CYTB/ND4 genes, or the absence of mutations in the ND2, ND5, ATP6, CYTB, ND4, ND1, COX3, 12S genes and an «unfavorable» classification according to the ELN score, is the indication of an unfavorable survival prognosis;
either the presence of at least one mutation in one of the ND2/ND5/ATP6/CYTB/ND4 genes and of at least one mutation in one of the ND1/COX3/12S genes, or the absence of mutations in the ND2, ND5, ATP6, CYTB, ND4, ND1, COX3, 12S genes and an «intermediate» classification according to the ELN score, is the indication of an intermediate survival prognosis.

7. The method according to claim 1, wherein:
either the presence of at least one mutation in one of the ND2/ND5/ATP6/CYTB/ND4 genes and the absence of mutations in the ND1/COX3/12S genes, or the absence of mutations in the ND2/ND5/ATP6/CYTB/ND4/ND1/COX3/12S genes and the presence of at least one mutation in one of the ND3/ATP8 genes and the absence of mutations in the COX1/COX2 genes, is the indication of a favorable survival prognosis;
either the presence of at least one mutation in one of the ND1/COX3/12S genes and the absence of mutations in the following ND2/ND5/ATP6/CYTB/ND4 genes, or the absence of mutations in the ND2/ND5/ATP6/CYTB/ND4/ND1/COX3/12S genes and the presence of at least one mutation in one of the COX1/COX2 genes and the absence of mutations in the ND3/ATP8 genes, is the indication of an unfavorable survival prognosis;
either the presence of at least one mutation in one of the ND2/ND5/ATP6/CYTB/ND4 genes and of at least one mutation in one of the ND1/COX3/12S genes, or the absence of mutations in the ND2/ND5/ATP6/CYTB/ND4/ND1/COX3/12S genes and the presence of at least one mutation in one of the COX1/COX2 genes and the presence of at least one mutation in one of the ND3/ATP8 genes, or the absence of mutations in the ND2/ND5/ATP6/CYTB/ND4/ND1/COX3/12S/COX1/COX2/ND3/ATP8 genes (Mitonaïve B/A patients), is the indication of an intermediate survival prognosis.

8. The method according to claim 1, wherein:
either the presence of at least one mutation in one of the ND2/ND3/ATP8/CYTB/ND4 genes and the absence of mutations in the COX1/COX2/COX3/12S genes, or the absence of mutations in the COX1/COX2/COX3/12S/ND2/ND3/ATP8/CYTB/ND4 genes but the presence of at least one mutation in one of the ATP6/ND5 genes and the absence of mutations in the ND1 gene, is the indication of a favorable survival prognosis;
either the presence of at least one mutation in one of the COX1/COX2/COX3/12S genes and the absence of mutation in the ND2/ND3/ATP8/CYTB/ND4 genes, or the absence of mutations in the COX1/COX2/COX3/12S/ND2/ND3/ATP8/CYTB/ND4 genes but the presence of at least one mutation in the ND1 gene and the absence of mutations in the ATP6/ND5 genes, is the indication of an unfavorable survival prognosis;
either the presence of at least one mutation in one of the ND2/ND3/ATP8/CYTB/ND4 genes and of at least one mutation in one of the COX1/COX2/COX3/12S genes, or the absence of mutations in the ND2/ND3/ATP8/CYTB/ND4/COX1/COX2/COX3/12S genes and the presence of at least one mutation in the ND5/ATP6 genes and the presence of at least one mutation in the ND1 gene, or the absence of mutations in the ND2/ND3/ATP8/CYTB/ND4/COX1/COX2/COX3/12S/ND5/ATP6/ND1 genes (Mitonaïve A/B patients), is the indication of an intermediate survival prognosis.

9. The method according to claim 7, wherein the method further comprises determining the ELN prognostic score in the subgroup of Mitonaïve B/A or Mitonaïve A/B patients.

10. The method according to claim 9, wherein:
either the presence of at least one mutation in one of the ND2/ND5/ATP6/CYTB/ND4 genes, and the absence of mutations in the ND1/COX3/12S genes, or the absence of mutations in the ND2/ND5/ATP6/CYTB/ND4/ND1/COX3/12S genes and the presence of at least one mutation in one of the ND3/ATP8 genes and the absence of mutations in the COX1/COX2 genes, or Mitonaïve B/A patients classified favorable with the classification of the ELN, is the indication of a favorable survival prognosis;
either the presence of at least one mutation in one of the ND1/COX3/12S genes, and the absence of mutations in the ND2/ND5/ATP6/CYTB/ND4 genes, or the absence of mutations in the ND2/ND5/ATP6/CYTB/ND4/ND1/COX3/12S genes and the presence of at least one mutation in one of the COX1/COX2 genes and the absence of mutations in the ND3/ATP8 genes, or Mitonaïve B/A patients classified unfavorable with the classification of the ELN, is the indication of an unfavorable survival prognosis;
either the presence of at least one mutation in one of the ND2/ND5/ATP6/CYTB/ND4 genes and at least one mutation in one of the ND1/COX3/12S genes or the absence of mutations in the ND2/ND5/ATP6/CYTB/ND4/ND1/COX3/12S genes and the presence of at least one mutation in one of the COX1/COX2 genes and the presence of at least one mutation in one of the ND3/ATP8 genes, or Mitonaïve B/A classified intermediate with the classification of the ELN, is the indication of an intermediate prognosis.

11. The method according to claim 9, wherein:
either the presence of at least one mutation in one of the ND2/ND3/ATP8/CYTB/ND4 genes and the absence of mutations in the COX1/COX2/COX3/12S genes, or the absence of mutations in the COX1/COX2/COX3/12S/ND2/ND3/ATP8/CYTB/ND4 genes but the presence of at least one mutation in one of the ATP6/ND5 genes and the absence of mutations in the ND1 gene, or the Mitonaïve A/B patients classified favorable with the classification of the ELN, is the indication of a favorable survival prognosis;
either the presence of at least one mutation in one of the COX1/COX2/COX3/12S genes and the absence of mutations in the ND2/ND3/ATP8/CYTB/ND4 genes, or the absence of mutations in the COX1/COX2/COX3/12S/ND2/ND3/ATP8/CYTB/ND4 genes but the presence of at least one mutation in the ND1 gene and the absence of mutations in the ATP6/ND5 genes, or the Mitonaïve A/B patients classified unfavorable with the classification of the ELN, is the indication of an unfavorable survival prognosis;
either the presence of at least one mutation in one of the ND2/ND3/ATP8/CYTB/ND4 genes and of at least one mutation in one of the COX1/COX2/COX3/12S genes, or the absence of mutations in the ND2/ND3/ATP8/CYTB/ND4/COX1/COX2/COX3/12S genes, and the presence of at least one mutation in the ND5/ATP6 genes and the presence of at least one mutation in the ND1 gene or the Mitonaïve A/B patients classified intermediate with the classification of the ELN, is the indication of an intermediate survival prognosis.

12. The method according to claim 1, wherein the detection steps are carried out by high-throughput sequencing of the mitochondrial genome.

13. An In vitro method for predicting or evaluating the effectiveness and/or benefit of a treatment of a chemosensitive cancer in a patient suffering from said cancer comprising the following steps of:
determining a survival prognosis according to a method as defined according to claim 1 from a biological sample of said patient before treatment;
determining a survival prognosis according to a method as defined according to claim 1 from a biological sample of said patient after treatment;
comparing survival prognoses before and after treatment;
determining either resistance to treatment when the survival prognosis of said patient after treatment is identical to or worse than the survival prognosis of said patient before treatment, or sensitivity to treatment when the survival prognosis of said patient after treatment is better than the survival prognosis of said patient before treatment.

14. The method according to claim 13, wherein the treatment of a chemosensitive cancer is a treatment with venetoclax.

15. The method according to claim 1, wherein the chemosensitive cancer is chosen from the group consisting of acute myeloid leukemias (AML), sarcomas, testicular cancer, choriocarcinomas, hemopathies, gynecological cancers, lung cancers, neuroblastomas, malignant brain tumors, digestive cancers, pancreatic cancers, bladder cancers, prostate cancers, thyroid cancers, liver cancers, ENT cancers.

16. A computer program comprising instructions, which when the program is executed by a computer, lead it to implement a method according to claim 1.

17. A computer-readable data medium including instructions, which when executed by a computer, lead it to implement a method according to claim 1.

18. A method comprising applying the—method according to claim 1, as a companion test.
